(19) United States
(12) Patent Application Publication
(10) Pub. No.: US 2025/0152483 A1
(43) Pub. Date: May 15, 2025

(54) TREATMENT AND PREVENTION OF DENTAL DEMINERALIZATION

(71) Applicant: The Regents of the University of Michigan, Ann Arbor, MI (US)

(72) Inventors: Brian Clarkson, Ann Arbor, MI (US); Nader Almutairi, Ann Arbor, MI (US); Sywe-Ren Chang, Ann Arbor, MI (US)

(21) Appl. No.: 18/839,931

(22) PCT Filed: Feb. 21, 2023
(86) PCT No.: PCT/US2023/062928

§ 371 Date: Aug. 20, 2024

Related U.S. Application Data
(60) Provisional application No. 63/312,247, filed on Feb. 21, 2022.

(51) Int. Cl. A61K 8/24; A61K 8/21; A61Q 11/00

(52) U.S. Cl. CPC A61K 8/24; A61K 8/21; A61Q 11/00; A61K 2800/412; A61K 2800/413

(57) ABSTRACT

The present disclosure provides methods for mineralizing or enhancing remineralization or treating or preventing demineralization (e.g., as a result of dental decay or caries) of a dental surface or subsurface. More particularly, the present disclosure provides methods comprising contacting the dental surface or oral cavity with an effective amount of fluorapatite crystals or a composition comprising fluorapatite crystals. The present disclosure also provides for dental care composition comprising fluorapatite crystals.

### CROSS-REFERENCE TO RELATED APPLICATIONS

This application claims the benefit of U.S. Provisional Application No. 63/312,247, filed Feb. 21, 2022, the content of which is herein incorporated by reference in its entirety.

### FIELD

The present invention relates to methods for remineralization of non-cavitated tooth surfaces.

### BACKGROUND

Caries is considered the most prevalent chronic disease affecting 95% of the world's population at some stage during their lifetime. Caries (dental decay) forms when bacteria in the dental biofilm on a tooth surface ferment sugars and produce acids, which demineralize the apatite tooth structures of teeth, enamel and/or dentin. Characteristics of carious lesions and decalcification in enamel are a white (when dried) roughened surface. Both of these characteristics indicate an increase in the microscopic pore size of the enamel and considered active lesions. Commonly, inactive lesions require no intervention while active lesions do. Early active lesions permit conservative remineralization treatment, while cavitated lesions require dental restoration. An active carious lesion is one that is advancing and has a slightly demineralized (˜5% compared to sound enamel) microporous surface, overlying a subsurface lesion that may have a porosity as high as 30%- 40%. The demineralization and remineralization process occurs numerous times daily, leading either to cavitation, repair, reversal, or maintenance of the existing state. If demineralization proceeds, irreversible cavitation may occur, necessitating a dental procedure to avoid progression and, eventual, pain and extraction of the tooth.

### SUMMARY

Provided herein are methods of mineralizing or enhancing remineralization of a dental (e.g., tooth) surface or subsurface. Also provided are methods treating or preventing demineralization of a dental surface or subsurface. In some embodiments, the methods comprise, consist of, or consist essentially of: contacting the dental surface or oral cavity of a subject with an effective amount of fluorapatite crystals or a composition comprising fluorapatite crystals.

In some embodiments, the dental surface comprises white spot carious lesions. In some embodiments, the dental subsurface comprises subsurface enamel. In some embodiments, the dental subsurface comprises a subsurface lesion.

In some embodiments, the fluorapatite crystals are between 10 nm and 300 nm in size. In some embodiments, the fluorapatite crystals are between 20 nm and 50 nm in size. In some embodiments, the fluorapatite crystals are between 100 nm and 300 nm in size.

In some embodiments, the fluorapatite crystals are rod shaped. In some embodiments, the fluorapatite crystals have a cross section of about 10 nm and a length of between 100 nm and 300 nm or between 20 nm and 50 nm.

In some embodiments, the fluorapatite crystals are provided at a concentration of 1-60%.

In some embodiments, the contacting is carried out daily, weekly, or monthly. In some embodiments, the contacting is for at least 7 days.

In some embodiments, the fluorapatite crystals or the composition comprising fluorapatite crystals is formulated as a gel, a solution, an emulsion, a suspension, an ointment, a paste, a cream, a powder, an acrosol, a spray, an oil, or a patch.

In some embodiments, the fluorapatite crystals further comprise a cargo molecule.

In some embodiments, the method does not include contacting the dental surface or oral cavity with dental cements, scaffolds, or sealants. In some embodiments, the method comprises contacting the dental surface or oral cavity with dental cements, scaffolds, or sealants. In some embodiments, the dental cements, scaffolds, or sealants comprise the fluorapatite crystals described herein.

In some embodiments, the method does not include contacting the dental surface or oral cavity with other sources of calcium phosphate, other sources of fluoride, or combinations thereof.

In some embodiments, the demineralization is caused by caries.

Further provided herein is a dental care composition comprising 1-60% fluorapatite crystals.

In some embodiments, the fluorapatite crystals are between 10 nm and 300 nm in size. In some embodiments, the fluorapatite crystals are between 20 nm and 50 nm in size. In some embodiments, the fluorapatite crystals are between 100 nm and 300 nm in size.

In some embodiments, the fluorapatite crystals are rod shaped. In some embodiments, the fluorapatite crystals have a cross section of about 10 nm and a length of between 100 nm and 300 nm or between 20 nm and 50 nm.

In some embodiments, the fluorapatite crystals further comprise a cargo molecule.

In some embodiments, the dental care composition comprises a toothpaste, a liquid dentifrice, a tooth powder, a dental paste, a gingival massage cream, a mousse, a topical oral gel, a mouth rinse, a denture product, a mouth spray, a lozenge, an oral tablet, a troche, a gargle tablets, a dental patch, or a chewing gum.

Other aspects and embodiments of the disclosure will be apparent in light of the following detailed description.

### DETAILED DESCRIPTION

Remineralization of a demineralized (non-cavitated) tooth surface has been a challenge for dentists worldwide. Disclosed herein are methods for restoring demineralized tooth surface using a fluorapatite (calcium fluorophosphate) crystals. Use of the disclosed methods resulted in a newly formed mineral crystal surface more resistant to acid, and likely more difficult to demineralize, than the original carbonated hydroxyapatite mineral. The synthesized nano-fluorapatite crystals (nFA) may resemble or mimic the same shape, size, and composition as human enamel crystals. The nFA crystals were applied to the surface of white spot lesions after tagging them with a Flou-4 fluorescing material and, surprisingly, penetration into not only the surface but also the subsurface of the lesions was observed,

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the terms “administering,” “providing”, and “introducing,” are used interchangeably herein and refer to the placement of the compounds or compositions of the disclosure into a subject by a method or route which results in at least partial localization to a desired site. The compounds or compositions can be administered by any appropriate route which results in delivery to a desired location in the subject.

The term “contacting” as used herein refers to bring or put in contact, to be in or come into contact. The term “contact” as used herein refers to a state or condition of touching or of immediate or local proximity. Contacting a composition or agent to a target destination, may occur by any means of administration known to the skilled artisan.

As used herein, the term “preventing” refers to partially or completely delaying onset of a disease, disorder and/or condition; partially or completely delaying onset of one or more symptoms, features, or clinical manifestations of a particular disease, disorder and/or condition; partially or completely delaying onset of one or more symptoms, features, or manifestations of a particular disease, disorder and/or condition; partially or completely delaying progression from a disease, disorder and/or condition; and/or decreasing the risk of developing pathology associated with the disease, disorder and/or condition.

A “subject” or “patient” may be human or non-human and may include, for example, animal strains or species used as “model systems” for research purposes, such a mouse model as described herein. The subject may include males or females. Likewise, a subject may include either adults or juveniles (e.g., children). Moreover, a subject may mean any living mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one embodiment of the methods and compositions provided herein, the mammal is a human.

As used herein, “treat,” “treating” and the like means a slowing, stopping, or reversing of progression of a disease, disorder, condition, or status when provided the compounds or compositions described herein to an appropriate subject. The term also means a reversing of the progression of such a disease, disorder, or condition. As such, “treating” means an application or administration of the methods described herein to a subject, where the subject has a disease or a symptom of a disease, disorder, condition, or status where the purpose is to cure, heal, alleviate, relieve, alter, remedy, ameliorate, improve, or affect the disease or symptoms of the disease, disorder, condition, or status.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Methods of Treatment

The present disclosure provides methods for mineralizing or enhancing remineralization or treating or preventing demineralization of a dental surface or subsurface (e.g., subsurface enamel). The methods comprise, consist of, or consist essentially of: contacting the dental surface or oral cavity with an effective amount of fluorapatite crystals or a composition comprising fluorapatite crystals.

The methods are not limited by the etiology of the demineralization. In some embodiments, the demineralization is a result of dental decay or caries. Thus, also provided herein are methods for treating or preventing dental decay or caries. In some embodiments, the dental surface comprises white spot carious lesions. In some embodiments, the dental subsurface comprises a subsurface lesion.

In some embodiments, the methods do not include contacting the dental surface or oral cavity with dental cements, scaffolds, or sealants. In some embodiments, the method comprises contacting the dental surface or oral cavity with dental cements, scaffolds, or sealants. In some embodiments, the dental cements, scaffolds, or sealants comprise the fluorapatite crystals described herein.

In addition, in some embodiments, the methods do not include contacting the dental surface or oral cavity with other sources of calcium, phosphate, fluoride, or combinations thereof. The calcium source may be from any inorganic calcium compound or organic calcium compound and the phosphate source may be from any phosphate compounds. Suitable single sources of calcium and phosphate ions include, but are not limited to, dicalcium phosphate anhydrous, tetracalcium phosphate, dicalcium phosphate dihydrate, tricalcium phosphate, and mixtures thereof. Suitable separate sources of the calcium ions and phosphate ions are, for example, calcium chloride, calcium sulfate, calcium aluminosilicate, calcium carbonate, calcium ascorbate, calcium oxide, calcium hydroxide, calcium lactate, calcium citrate, or calcium gluconate, as calcium source and sodium phosphate or potassium phosphate as a phosphate source. Suitable fluoride sources include an alkali metal fluoride, an alkali metal monofluorophosphate, stannous fluoride or an amine fluoride.

The fluorapatite crystals may resemble in shape, size, and composition enamel crystals. For example, in some embodiments, the fluorapatite crystals may range in size from 20-50 nm (e.g., about 20 nm, about 30 nm, about 40 nm or about 50 nm). In some embodiments, the fluorapatite crystals are between 10 nm and 300 nm in size. The fluorapatite crystals may be between 10 nm and 300 nm, 10 nm and 250 nm, 10 nm and 200 nm, 10 nm and 150 nm, 10 nm and 100 nm, 10 nm and 50 nm, 10 nm and 25 nm, 25 nm and 300 nm, 25 nm and 250 nm, 25 nm and 200 nm, 25 nm and 150 nm, 25 nm and 100 nm, 25 nm and 50 nm, 50 nm and 300 nm, 50 nm and 250 nm, 50 nm and 200 nm, 50 nm and 150 nm, 50 nm and 100 nm, 100 nm and 300 nm, 100 nm and 250 nm, 100 nm and 200 nm, 100 nm and 150 nm, 150 nm and 300 nm, 150 nm and 250 nm, 150 nm and 200 nm, 200 nm and 300 nm, 200 nm and 250 nm, and 250 nm and 300 nm in size. In select embodiments, the fluorapatite crystals are between 100 nm and 300 nm in size.

In some embodiments, the fluorapatite crystals are rod shaped. In some embodiments, the fluorapatite crystals have a cross section of about 10 nm. In some embodiments, the fluorapatite crystals have a cross section of about 10 nm and have a length of between 100 nm and 300 nm. In some embodiments, the fluorapatite crystals have a cross section of about 10 nm and have a length of between 20 nm and 50 nm.

Compositions that can be applied locally to the dental surface or oral cavity may be in any form including solids (e.g., powders), solutions, oils, creams, ointments, gels, emulsions, suspensions, lotions, pastes, foams, sprays, aerosols, patches, and the like. The use of such carriers for pharmaceutically active substances is well known in the art. The compositions and methods for their preparation will be readily apparent to those skilled in the art. Techniques and formulations may be found, for example, in Remington's Pharmaceutical Sciences, 19th Edition (Mack Publishing Company, 1995).

In some embodiments, the fluorapatite crystals are added to or mixed with dental care compositions.

The specific amount and frequency or duration of administration of the fluorapatite crystals may depend upon a variety of factors including the location and severity of the dental decay or lesions. An “effective amount” is an amount that is delivered, either in a single administration or as part of a series, which achieves the desirable effect, e.g., mineralizing or enhancing remineralization of lesions.

In some embodiments, the fluorapatite crystals are provided at a concentration of 1-60%. For example, the fluorapatite crystals are provided at a concentration of about 1%, about 5%, about 10%, about 15%, about 20%, about 25%, about 30%, about 35%, about 40%, about 45%, about 50%, about 55%, or about 60%. In some embodiments, the fluorapatite crystals are provided at a concentration of 1-40%, 1-25%, 1-10%, 1-5%, 5-15%, 5-25%, 5-40%, 5-60%, 10-20%, 10-40%, 10-60%, 20-40%, 20-60%, or 40-60%.

In some embodiments, the effective amount is delivered daily, either as a single administration or multiple administrations throughout the day. In some embodiments, the effective amount is delivered more than once daily, for example every 6-8 hours or 2-4 times daily. In some embodiments, the effective amount is delivered less than once daily, for example, every 2 days, twice weekly, once weekly, or monthly.

In some embodiments, the fluorapatite crystals or the composition comprising fluorapatite is administered for one to four weeks or more. In some embodiments, the fluorapatite crystals or the composition comprising fluorapatite is administered for about 1 week, about 2 weeks, about 3 weeks, about 4 weeks, about 5 weeks, about 6 weeks, about 7 weeks, about 8 weeks, about 12 weeks, or about 16 weeks.

In some embodiments, the fluorapatite crystals further comprise a cargo molecule. The cargo may comprise an active agent. In some embodiments, the active agent comprises a therapeutic agent, a contrast agent, a detectable marker or label, or any combination thereof. “Active agent” as used herein refers to any compound useful for therapeutic, prophylactic, or diagnostic purposes (e.g., any compound that is administered to a subject for the treatment, prevention, or diagnosis of a condition).

In some embodiments, the active agent comprises a detectable marker or label. It will be understood that a label contemplated by the disclosure includes chemiluminescent molecules, radioactive labels, dyes, fluorescent molecules (e.g., small synthetic compounds or fluorescent proteins), and phosphorescent molecules, as well as other detectable labels known in the art.

### Dental Care Composition

The present disclosure provides a dental care composition comprising fluorapatite crystals. In some embodiments, the dental care composition comprises 1-60% fluorapatite crystals. For example, the dental care composition comprises about 1%, about 5%, about 10%, about 15%, about 20%, about 25%, about 30%, about 35%, about 40%, about 45%, about 50%, about 55%, or about 60% fluorapatite crystals. In some embodiments, the dental care composition comprises 1-40%, 1-25%, 1-10%, 1-5%, 5-15%, 5-25%, 5-40%, 5-60%, 10-20%, 10-40%, 10-60%, 20-40%, 20-60%, or 40-60% fluorapatite crystals.

The fluorapatite crystals may resemble in shape, size, and composition enamel crystals. For example, in some embodiments, the fluorapatite crystals may range in size from 20-50 nm (e.g., about 20 nm, about 30 nm, about 40 nm or about 50 nm). However, in some embodiments, the fluorapatite crystals are between 10 nm and 300 nm in size. The fluorapatite crystals may be between 10 nm and 300 nm, 10 nm and 250 nm, 10 nm and 200 nm, 10 nm and 150 nm, 10 nm and 100 nm, 10 nm and 50 nm, 10 nm and 25 nm, 25 nm and 300 nm, 25 nm and 250 nm, 25 nm and 200 nm, 25 nm and 150 nm, 25 nm and 100 nm, 25 nm and 50 nm, 50 nm and 300 nm, 50 nm and 250 nm, 50 nm and 200 nm, 50 nm and 150 nm, 50 nm and 100 nm, 100 nm and 300 nm, 100 nm and 250 nm, 100 nm and 200 nm, 100 nm and 150 nm, 150 nm and 300 nm, 150 nm and 250 nm, 150 nm and 200 nm, 200 nm and 300 nm, 200 nm and 250 nm, and 250 nm and 300 nm in size. In select embodiments, the fluorapatite crystals are between 100 nm and 300 nm in size.

In some embodiments, the fluorapatite crystals are rod shaped. In some embodiments, the fluorapatite crystals have a cross section of about 10 nm. In some embodiments, the fluorapatite crystals have a length of between 100 nm and 300 nm. In some embodiments, the fluorapatite crystals have a length of between 20 nm and 50 nm.

“Dental care composition” refers to a product which in the ordinary course of usage is not intentionally swallowed for purposes of systemic administration of a particular agents but is rather retained in the oral cavity for a time sufficient to contact substantially all of the dental surfaces and/or oral tissues for the intended purpose. The dental care composition of the present invention may be in the form of toothpaste, liquid dentifrice, tooth powder, dental paste, gingival massage cream, mousse, topical oral gel, mouth rinse, denture product, mouth spray, lozenge, oral tablet, troche, gargle tablet, dental patch, or chewing gum.

The dental care composition disclosed herein may further include additional well known ingredients depending on the type and form of a particular oral composition.

In some embodiments, the fluorapatite crystals further comprise a cargo molecule. The cargo may comprise an active agent. In some embodiments, the active agent comprises a biomolecule, a therapeutic agent, a contrast agent, a detectable marker or label, or any combination thereof.

In some embodiments, the active agent comprises a detectable marker or label. It will be understood that a label contemplated by the disclosure includes chemiluminescent molecules, radioactive labels, dyes, fluorescent molecules (e.g., small synthetic compounds or fluorescent proteins), and phosphorescent molecules, as well as other detectable labels known in the art.

In some embodiments, the disclosed dental care composition may be used for diagnosis of lesions, dental demineralization, or other conditions characterized by dental decay.

### EXAMPLES

The following are examples of the present invention and are not to be construed as limiting.

### Example 1

Permanent human caries free teeth, extracted for orthodontic or periodontal reasons, were used in this study. Teeth were cleaned with ultrasonic scaler and stored in sodium azide.

Samples were sectioned longitudinally using Buehler Isomet low-speed saw. Enamel windows 3×3 mm were created on the buccal surface of the teeth using adhesive UPVC tape, the remaining tooth surfaces were coated by acid resistant varnish (colored nail polish). Artificial demineralization was carried out using an acidic gel. Sound enamel specimens were demineralized at pH 4.6 and 37° C. in 10% methylcellulose (aqueous, 1,500 cP, 63 kDa), coated with an equal volume of 0.1 M lactic acid, pH adjusted with KOH, for seven days. Sodium azide (3 mM) was added to all demineralization solutions as a bacteriostat. After demineralization, specimens were rinsed with deionized water. Then, stored separately in Eppendorf vials containing a moist cotton pellet at room temperature.

To synthesize nano fluorapatite crystals (nFA), hydroxyapatite powder (104.6 mg) and sodium fluoride (8.4 mg) were mixed with 100 mL of distilled water. The suspensions were agitated continuously, and HNO₃ was added until the powder dissolved. After that, the pH was adjusted to 2.4. Ammonium hydroxide was then added dropwise to 20 mL of the preceding solution with continuous stirring until the desired pH of 6 was reached. The suspension was sealed in a plastic tube and kept in a water bath at 70° C. for three days.

Fluo-4 pentapotassium salt (F14200) was purchased from Invitrogen and stored in the dark at −20° C. The dye was thawed to room temperature for working solutions and then kept in the dark at 4° C. The potassium salt of this dye was used to provide water solubility.

Samples were subjected to Micro C.T scan to measure the depth of the created enamel lesion before and after treatment. They were placed in a 19 mm diameter specimen holder and scanned over the tooth's entire length using a micro-CT system (μCT100 Scanco Medical, Bassersdorf, Switzerland). Scan settings were voxel size 10 μm, 90 kVp, 44 μA, 0.5 mm AL filter, and integration time 500 ms. The analysis was completed using the manufacturer's evaluation software to measure the depth of the lesion in 3 locations spaced equidistantly across the width of the defect.

After micro-CT scanning, staining of nFA with Fluo-4 was done according to Hernandez-Santana et al. (Bioanalysis 3(10): 1085-91 2011, incorporated herein by reference in its entirety). Fluo-4 (20 μl, 1×10⁻³ M) was added to the Eppendorf tube which has 5 mg (5%) of nFA and mixed at room temperature for 60 min. Samples were always covered from light. The sample was then centrifuged at 9000 rpm for 1 min and the supernatant removed. The supernatant was removed, the pellet resuspended in water (100 μl).

Three carious lesions were exposed to the nFA tagged with Fluo-4 at a concentration of 5% for 30 minutes. The control was DI-water treated teeth for the same time periods. Treated teeth were then washed in DI-water for 2 mins to remove non-adsorbed material. Exposed teeth were illuminated using a fluorescent microscope and pictures were taken to show fluorescence. The specimens were examined using a Leica TCS SP8 2-Photon Confocal using a 40× oil objective. Samples placed on a glass-bottomed petri dish. The multi-photon illumination wavelength used was set to 960 nm and z-stack images collected and analyzed.

Micro-CT results after demineralization showed a mean depth of 0.176 mm. A difference between the control and the experimental group was noticed when samples were exposed to light curing unit showing an illuminance of the nFA crystals within the lesion. A pictures of 2-photon microscopy showed crystals fluorescence and z-stack images were obtained showing the extent of the nFA crystals inside the lesion.

### Example 2

Extracted permanent human teeth sectioned longitudinally. Enamel windows 3×3 mm were created. Artificial demineralization was carried out using acidic gel pH 4.6 for 7 days, as described in Example 1. Nano fluorapatite crystals were synthesized as described in Example 1. Fluo-4 pentapotassium salt was used to tag the crystals and be able to illuminate when exposed to fluorescent microscopy. Micro C.T scan used to measure the depth of the enamel lesion before and after treatment. Carious lesions were exposed to the nFA tagged with Fluo-4 at a concentration of 15% for a period of 30 mins. The control was DI-water treated teeth for the same time periods and Fluo-4 solution. The treated teeth washed in DI-water for 2 mins. Exposed teeth were illuminated by a Light curing unit and pictures were taken to show fluorescence. The specimens were examined using a Leica TCS SP8 2-Photon Confocal using a 40× oil objective to verify presence of nFA crystals and the depth of penetration.

Micro-CT results after demineralization showed an average depth of lesions of 150 um. A difference between the control and the experimental group was noticed when samples were exposed to light curing unit showing an illuminance of the nFA crystals within the lesion. A pictures of 2-photon microscopy showed crystals fluorescence and z-stack images were obtained showing the extent of the nFA crystals inside the lesion.

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

What is claimed is:

1. A method of mineralizing or enhancing remineralization of a dental surface or subsurface comprising contacting the dental surface or oral cavity of a subject with an effective amount of fluorapatite crystals or a composition comprising fluorapatite crystals.

2. A method of treating or preventing demineralization of a dental surface or subsurface comprising contacting a dental surface or oral cavity of a subject with an effective amount of fluorapatite crystals or a composition comprising fluorapatite crystals.

3. The method of claim 1 or claim 2, wherein the dental surface comprises white spot carious lesions.

4. The method of any of claims 1-3, wherein the dental subsurface comprises subsurface enamel.

5. The method of any of claims 1-4, wherein the dental subsurface comprises a subsurface lesion.

6. The method of any of claims 1-5, wherein the fluorapatite crystals are between 10 nm and 300 nm in size.

7. The method of claim 6, wherein the fluorapatite crystals are between 20 nm and 50 nm in size.

8. The method of claim 6, wherein the fluorapatite crystals are between 100 nm and 300 nm in size.

9. The method of any of claims 1-8, wherein the fluorapatite crystals are rod shaped.

10. The method of claim 9, wherein the fluorapatite crystals have a cross section of about 10 nm and a length of between 100 nm and 300 nm or between 20 nm and 50 nm.

11. The method of any of claims 1-10, wherein the fluorapatite crystals are provided at a concentration of 1-60%.

12. The method of any of claims 1-11, wherein the contacting is daily, weekly, or monthly.

13. The method of any of claims 1-12, wherein the contacting is for at least 7 days.

14. The method of any of claims 1-13, wherein the fluorapatite crystals or the composition comprising fluorapatite crystals is formulated as a gel, a solution, an emulsion, a suspension, an ointment, a paste, a cream, a powder, an aerosol, a spray, an oil, or a patch.

15. The method of any of claims 1-14, wherein the fluorapatite crystals further comprise a cargo molecule

16. The method of any of claims 1-15, wherein the method does not include contacting the dental surface or oral cavity with dental cements, scaffolds, or sealants.

17. The method of any of claims 1-16, wherein the method does not include contacting the dental surface or oral cavity with other sources of calcium phosphate, other sources of fluoride, or combinations thereof.

18. The method of any of claims 2-17, wherein the demineralization is caused by caries.

19. A dental care composition comprising 1-60% fluorapatite crystals.

20. The dental care composition of claim 19, wherein the fluorapatite crystals are between 20 nm and 300 nm in size.

21. The dental care composition of claim 20, wherein the fluorapatite crystals are between 20 nm and 50 nm in size.

22. The dental care composition of claim 20, wherein the fluorapatite crystals are between 100 nm and 300 nm in size.

23. The dental care composition of any of claims 19-22, wherein the fluorapatite crystals are rod shaped.

24. The dental care composition of claim 23, wherein the fluorapatite crystals have a cross section of about 10 nm and a length of between 100 nm and 300 nm.

25. The dental care composition of any of claims 19-24, wherein the fluorapatite crystals further comprise a cargo molecule.

26. The dental care composition of any of claims 19-25, wherein the dental care composition comprises a toothpaste, a liquid dentifrice, a tooth powder, a dental paste, a gingival massage cream, a mousse, a topical oral gel, a mouth rinse, a denture product, a mouth spray, a lozenge, an oral tablet, a troche, a gargle tablets, a dental patch, or a chewing gum.

27. Use of fluorapatite crystals in the manufacture of a medicament or composition for treating or preventing demineralization of a dental surface or subsurface, or mineralizing or enhancing remineralization of a dental surface or subsurface, or diagnosing lesions, dental demineralization, or other conditions characterized by dental decay.

28. Use fluorapatite crystals for mineralizing or enhancing remineralization of a dental surface or subsurface.
